(19) United States
(12) Patent Application Publication
(10) Pub. No.: US 2008/0009717 A1
(43) Pub. Date: Jan. 10, 2008

(54) Method for generating a medical image and a medical imaging system

(76) Inventors: Klaus Herrmann, Nuremberg (DE); Eike Rietzel, Darmstadt (DE)

Correspondence Address: Siemens Corporation;Intellectual Property Department, 170 Wood Avenue South, Iselin, NJ, 08830, US

(21) Appl. No.: 11/799,959

(22) Filed: May 3, 2007

Related U.S. Application Data
(60) Provisional application No. 60/798,264, filed on May 5, 2006.

(30) Foreign Application Priority Data
May 5, 2006 (DE) 10 2006 021 051.4

(51) Int. Cl. A61B 5/05

(52) U.S. Cl. 600/425

(57) ABSTRACT

The invention relates to a method for generating a medical image with at least one emission source and at least one detector of a medical imaging system, with the radiation from the emission source penetrating the object and attenuated by the object being detected by the detector to generate the medical image as a mapping of the object structure. The invention also relates to a medical imaging system for the generation of image data as a basis for the generation of a medical image. By the provision of a freely positionable emission source and a detector, the possibility of expanding the overlap area of the radiation thus detected up to the structural limits of the medical imaging system is provided. A volume image data record, e.g. a computer tomography (CT), thus forms the complete investigation object located between the emission source and the detector.

### CROSS REFERENCE TO RELATED APPLICATIONS

The present application claims the benefit of the provisional patent application filed on May 5, 2006, and assigned application No. 60/798,264. The present application also claims priority of German application No. 10 2006 021 051.4 filed on May 5, 2006. Both of the applications are incorporated by reference herein in their entirety.

### FIELD OF THE INVENTION

The invention relates to a method for generating a medical image with at least one emission source and at least one detector of a medical imaging system, with the radiation from the emission source penetrating the object and the attenuatable radiation from an object being detected by the detector to generate the medical image. The invention also relates to a medical imaging system for the generation of image data as a basis for creating a medical image.

### BACKGROUND OF THE INVENTION

From the prior art, imaging systems, e.g. computer tomography, based on x-ray devices are known. Thus DE 197 46 093 A1 discloses an imaging system with an x-ray radiation source and an x-ray radiation receiving device, with the x-ray system being used to record two-dimensional projections and then being used for three-dimension image reconstruction, with the projection angles required for the image reconstruction being determined by transmitting and receiving devices for soundwaves or electromagnetic waves.

The emission sources of conventional three-dimensional imaging systems are mainly x-ray sources that are also arranged the same as the correspondingly aligned detector relative to an isocenter. The axes of rotation of the emission source and the detector that rotates with it, usually offset 180°, have up to now been arranged so that these run through an imaginary line of the beam path from the emission source to the center of the detector. The overlap area of the possible beam path with this conventional imaging system forms a volume area as a “reconstruction volume” that is detected by the imaging system and is the basis for the succeeding 3D image processing in the form of a 3D data record. On the basis of a defined number of fluoroscopic images with preset angle distances during an orbital rotation of 1900 the “SIREMOBIL Iso-C3D” imaging system, for example, generates a cubiform three-dimensional reconstruction volume with an edge length of up to 12 cm (“Klinische Studie zur registrierungsfreien 3D-Navigation mit dem mobilen C-Bogen SIREMOBIL Iso-C3D” [Clinical Study for Registration-Free 3D-Navigation with the SIREMOBIL Iso-C^{3D} Mobile C-Arm], P. A. Grutzner, A. Hebecker, H. Waelti, B. Vock, L.-P. Nolte, A. Wentzensen, electromedica 71, Vol 1, Pages 58-67, 2003)

Due to this limitation of the three-dimensional reconstruction volume it has up to now been possible only either to enlarge the imaging system so that an enlarged volume of the object to be examined is fluoroscoped and therefore a correspondingly large volume image data record is generated or, as an alternative, it has been possible, particularly with mobile imaging systems such as the x-ray C-arm system, to align the imaging system to the new investigation region. However, this new alignment of the mobile imaging system to another investigation region of the object requires a certain amount of experience on the part of the operator and is impractical, particularly for monitoring the progress of an operation during said operation.

### SUMMARY OF THE INVENTION

The object of the invention is to provide a possibility of generating medical images that enable a complete imaging of the object under investigation in a volume image data record based on 2D x-ray systems, especially in oncology and angiography, as well as also on the basis of mobile imaging systems in surgery, without it being necessary to enlarge the structural dimensions, especially of mobile imaging systems.

This object is achieved according to the invention by a method for generating a medical image by mean of a medical imaging system that provides independent positioning of an emission source and of a detector of the medical imaging system for each object irradiation relative to one other, with at least part of the object under investigation being arranged between the emission source and the detector and at least one part of the radiation of the emission source being detected by the detector.

Within the meaning of the invention, an object is a volume body that can be investigated by a medical imaging system, especially the body of a human or animal patient.

After detection of the irradiation of the object by determining the partially attenuated radiation during the fluoroscopy of the object at a specific timepoint, the respective position of the emission source and of the detector is determined at the same time for each object irradiation. The knowledge of the exact position of the emission source and of the detector for each object irradiation relative to each other and in respect of the object is indispensable for the formation of the volume image data record, e.g. as a CT image data record, and therefore for the image reconstruction.

The totality of the object irradiations defines at least an overlap area as a three-dimensional reconstruction volume that corresponds to the investigation region of the object. The volume image data set is formed on the basis of the three-dimensional reconstruction volume taking account of the relative positions of the emission source and detector for each object irradiation. The medical image is then extracted from the volume data record, relative to at least one preset section plane, using known image processing methods.

A main advantage of the method according to the invention is that due to the independent positioning of the emission source and of the detector almost any volume can be chosen as the overlap area of the object irradiation and is limited only by the structural dimensions of the imaging system between the emission source and the detector. Fixed imaging systems in oncology and angiography and also mobile imaging systems in surgery conventionally have only one center of rotation of the imaging system because of the isocentric arrangement of the emission source and of the detector, and thus generate only a narrowly limited reconstruction volume. By means of the method according to the invention, the reconstruction volume, and therefore the volume image data record, are substantially enlarged. With the method according to the invention, multiple alignments of an imaging system, whether operated as a fixed or mobile system, are omitted and the size of the investigated region of the object is also enlarged at the same time, with the structural dimensions of the imaging system remaining unchanged.

In an advantageous embodiment of the method, image distortions of the object irradiation detected in the detector during the generation of the three-dimensional reconstruction volume are taken into account. Where the detector is offset relative to the direction of the respective central beam of the emission source, geometric image distortions occur, resulting in a faulty image. Because the reconstruction volume is the basis for forming the volume image data record, the image distortions influence the quality of the volume image data record and therefore also the quality of the medical images generated therefrom. The determination of these image distortions using the position of the emission source relative to the detector area is taken into account with the method according to the invention when generating the image data record associated with the reconstruction volume. By these measures, a positioning of the emission source relative to the detector which is less than optimum has no influence on the quality of the resulting image.

To avoid the occurrence of distortions during the superpositioning of the object irradiations, the distortions are advantageously taken into account during the generation of the three-dimensional reconstruction volume. By the use of an x-ray radiation source as the emission source, object irradiation attenuated by an object is determined in the detector in the form of an attenuation profile. The sum of the relevant object irradiation for an isocentric imaging system, for example, leads to a star-shaped distortion in the return projection and therefore to a corruption of the image data of the reconstruction volume. By taking these distortions into account during the generation of the reconstruction volume, e.g. by filtering and folding, these distortions are compensated for and therefore a high image quality of the medical images is guaranteed.

For optimum processing of the image data of the three-dimensional reconstruction volume and of the volume image data record, it is regarded as advantageous if the image data is depicted in the form of a projection matrix, with the projection matrix taking into account the volume of the complete object under investigation. “Truncation artifacts” can be compensated for in this way. Truncation artifacts depend on the angle of the image plane that is generated by an unequal signal scanning in two directions. This produces artificial signal intensities leading to geometric deviations and thus to a faulty image generation of the object under investigation. In particular, thickness conditions of the structure in the investigated region are incorrectly represented if a truncation artifact is present in the image data. By depicting the projection data of the three-dimensional reconstruction volume in an object-size projection matrix, this image data is used for the volume image data record of the complete object and truncation artifacts are thus avoided.

In an advantageous embodiment of the method, it is provided that the emission source and the detector are positioned independently of each other during the object irradiation so that the object irradiation forms an overlap area, and therefore a three-dimensional reconstruction volume of the size of the object and a volume image data record for the complete object can thus be generated on the basis of the three-dimensional reconstruction volume formed in that way.

Alternatively, it is provided that the emission source and the detector are arranged relative to each other during each object irradiation in such a way that the object irradiation can form at least two overlap areas and therefore at least two three-dimensional reconstruction volumes of the object. Advantageously, volume image data records are generated in each case on the basis of the three-dimensional reconstruction volumes, with the volume image data records thus formed being combined by means of graphic image data processing methods into a complete volume image data record. Alternatively, the three-dimensional reconstruction volumes are combined by means of graphic image data processing methods to form a complete three-dimensional reconstruction volume and the complete three-dimensional reconstruction volume formed in this way is used as a basis for the generation of a complete volume image data record. The combination of the image data records can thus either take place on the plane of the individually formed image data records or can be already combined on the plane of the three-dimensional reconstruction volume and thus combine to form one three-dimensional reconstruction volume.

Advantageously, the image data of the three-dimensional reconstruction volume and/or of the individual volume image data records are combined with a graphic image data processing method, e.g. an image stitching method. This procedure, applied particularly in digital image processing, is used to stitch together individual images of an object so that the individual images can be combined to form a complete picture of the object. For example, it is then possible to create panoramic pictures (e.g. of buildings) from individual images of an object with different recording angles. By using these graphic image data processing methods, image data records of the individual three-dimensional reconstruction volumes and/or of the individual volume image data records can be combined into corresponding complete image data records. This affords the possibility of imaging the complete object by means of one image data record and assessing the generation of section plane images relative to the complete volume of the object as medical images.

The invention also relates to a medical imaging system with at least one emission source for the generation of a beam of radiation and with a detector for detection of the radiation, with the radiation detected by the detector being used to generate a medical image.

According to the invention, it is provided that the relative position and alignment of the emission source and detector takes place relative to a selectable center of rotation by means of at least one positioning element, with a control being guaranteed that enables the emission source and the detector to be positioned so that at least part of the beam of the emission source strikes the detector. Advantageously, the positioning element can freely position the emission source and/or the detector, such as is for example possible by means of a multiaxis robot.

In an advantageous embodiment of the medical imaging system, the emission source and the detector are arranged on a C-arm and the C-arm can be moved vertically and/or horizontally by means of the positioning element.

Also, at least two positioning elements can be advantageously combined by at least one rotatably mounted coupling element and thus guarantee a free positioning of the emission source and of the detector relative to a freely selectable center of rotation.

### BRIEF DESCRIPTION OF THE DRAWINGS

Further advantageous measures are described in the remaining subclaims; the invention is described in more detail using exemplary embodiments and the following figures. The illustrations are as follows.

FIG. 1A schematic section view of the three-dimensional reconstruction volume based on the isocentric arrangement of the emission source and of the detector according to the prior art;

FIG. 2a, 2b A schematic section view of two three-dimensional reconstruction volumes based on the use of the method according to the invention;

FIG. 3 A schematic section view of a three-dimensional reconstruction volume of the size of the object under investigation based on the free positioning of the emission source and of the detector according to the inventive method;

FIG. 4 A schematic representation of the method according to the invention;

FIG. 5 A representation of the formation of the projection matrix according to the inventive method on the basis of the evaluation of three object irradiations;

FIG. 6 A schematic representation of a mobile imaging system according to the invention with an isocentric C-arm that can be adjusted vertically and horizontally;

FIG. 7 A schematic representation of an imaging system according to the invention with rotatably mounted positioning elements.

In the figures, features that are the same are given the same character reference numbers.

### DETAILED DESCRIPTION OF THE INVENTION

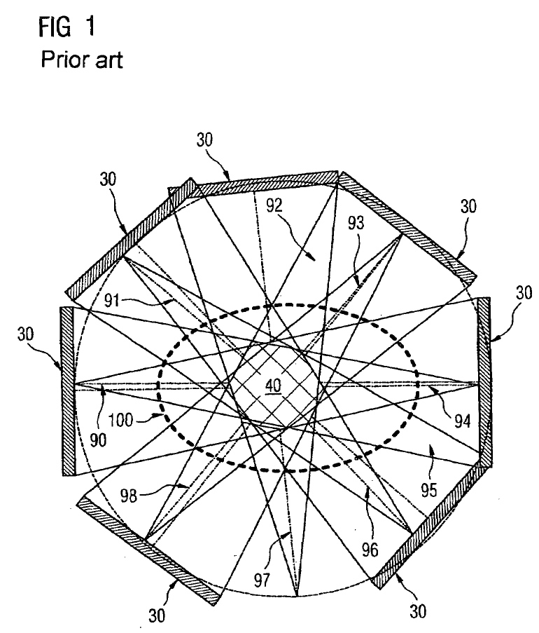

FIG. 1 shows the prior art. Conventionally, the emission source 20 (not illustrated) and the detector 30 are rotatably arranged in the form of a surface around an isocenter. This causes a movement of the emission source 20 and of the detector 30 on an orbit, shown by a peripheral broken line in FIG. 1. The emission source 20 generates a beam and emits this at different angles, each of which is detected as object irradiation 90 to 96 by the corresponding detector 30 that rotates with it. The beam pattern is shown in the shape of a cone with the emission source 20 (not illustrated) generating the beam in the acute angle of the beam path of the object irradiations 90 to 98. The overlap area of these object irradiations 90 to 96 close to the isocenter forms the three-dimensional reconstruction volume 40, with it also being necessary to allow beam paths to pass through outside the drawing plane of the object 100 in order to form the three-dimensional reconstruction volume 40.

For a better representation, only some selected object irradiations 90 to 99 in the drawing plane are shown in the drawing. To generate a volume image data record, image data is required from a number of beam paths from different recording positions and angles, so that in this case the illustrated object irradiations 90 to 99 are merely to be regarded as selected representative beam paths for a better description of the inventive idea.

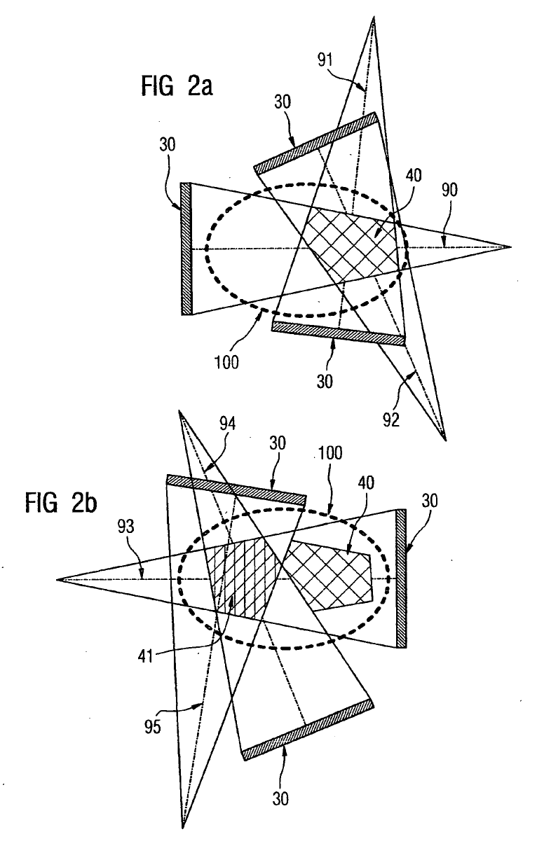

FIGS. 2a and 2b show a visualization of the generation of two three-dimensional reconstruction volumes 40, 41 according to the inventive method. The object 100 is fluoroscoped by the object irradiation 90 to 99 by a free positioning of the emission source 20 (not illustrated) and of the detector 30. Furthermore, in a first step the emission source 20 and the detector 30 are positioned around the object in such a way that a first three-dimensional reconstruction volume 40 is generated by the corresponding object irradiations 90 to 92 (FIG. 2a). In a second step according to the inventive method, the emission source 20 and the detector 30 are aligned relative to a second investigation region and thus form a second three-dimensional reconstruction volume 41 (FIG. 2b) that deviates from the first three-dimensional reconstruction volume 40 within the object 100. These three-dimensional reconstruction volumes 40, 41 can be either combined directly with each other by means of image data processing methods and then combined to form a volume image data record 50 (not illustrated) or alternatively the three-dimensional reconstruction volumes 40, 41 can serve as a basis for forming two volume image data records 50, 51 (not illustrated) that can then be combined into one complete volume image data record 50.

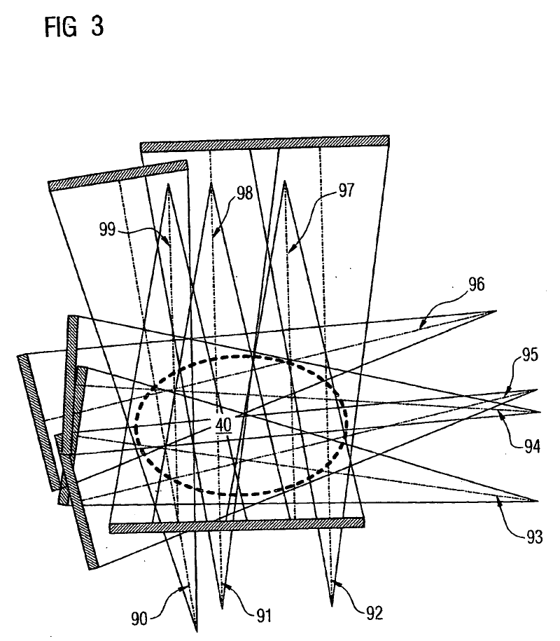

FIG. 3 shows a schematic section view of a three-dimensional reconstruction volume 40 of the size of the object 100 under investigation based on the free positioning of the emission source 20 (not illustrated) and of the detector 30 according to the inventive method. Due to the complete coverage of the volume of the object 100 with the object irradiations 90 to 99, in contrast to the conventional prior art, not only does a three-dimensional reconstruction volume 40 result in the immediate vicinity of the isocenter but also a three-dimensional reconstruction volume 40 of the size of the object 100 can be generated. Only the distance between the emission source 20 and the detector 30 limits the possible size of the three-dimensional reconstruction volume 40 corresponding to the inventive method.

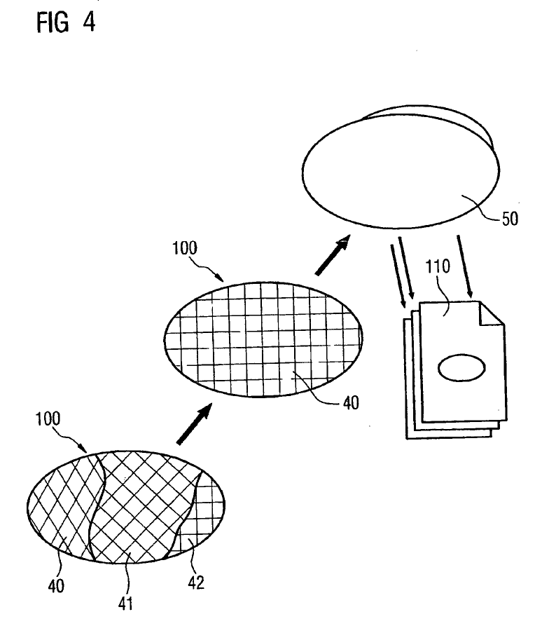

In the flow diagram shown in FIG. 4, the essential steps of the inventive method for the generation of several three-dimensional reconstruction volumes 40 to 42 are shown with the succeeding image processing. By means of a suitable positioning of the emission source 20 (not illustrated) and of the detector 30 (not illustrated), the object to be investigated 100 is completely covered by object irradiations 90 to 99 (not illustrated), with the overlapping areas of the object irradiations 90 to 99 in the illustrated example generating three three-dimensional reconstruction volumes 40, 41, 42. By means of an image data processing method, such as the stitching method, the image data of the reconstruction volumes 40, 41, 42 is combined to form one reconstruction volume 40 relative to the complete object 100. This image data of the reconstruction volume 40 then serves as a basis for the generation of the volume image data record 50, from which the medical images 110 can be extracted from the volume image data record 50 as plane section images. During the image processing of the image data of the three-dimensional reconstruction volumes 40, 41, 42, geometric distortion during the recording of the object irradiations 90 to 99 by the detector 30 and deformations during the superpositioning of the image data of the object irradiations 90 to 99 on one of the reconstruction volumes 40, 41, 42 are also taken into account and compensated for during the image data processing.

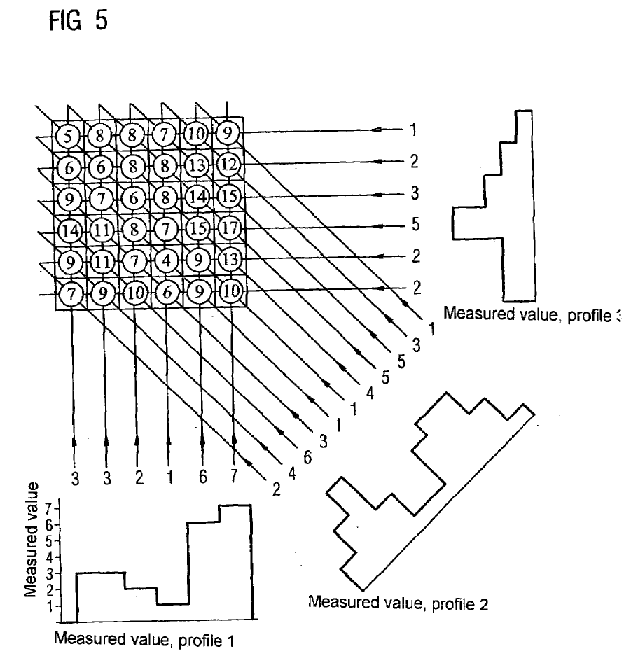

FIG. 5 is an illustration of the formation of the projection matrix according to the inventive method on the basis of the evaluation of three object irradiations 90-92 (not illustrated). The measured profiles of three object irradiations 90 to 92, detected in the detector 30 (not illustrated), are projected back into the projection matrix, taking account of the respective detection angle. The individual measured profiles show the amount of the attenuation of the radiation emitted from the emission source 30 (not illustrated) and attenuated by the specific structure of the object 100 (not illustrated). Ideally, the projection matrix depicts the complete volume of the object 100. Possible distortions and deformations that can lead to an impairment of the measured profiles are also taken into account during the image processing in the projection matrix, e.g. by location-specific filter algorhythms.

FIG. 6 shows a schematic representation of a mobile imaging system 10 according to the invention. A conventional C-arm 60 with a fixed connected emission source 20 and a fixed connected detector 30 can be rotated by means of a C-arm positioning element 80 relative to an isocenter (not illustrated) about the orbital axis of the C-arm 60. The C-arm 60 is connected to two positioning elements and can be moved vertically and/or horizontally by means of the positioning elements 70. Because of the possibility of the vertical and/or horizontal movement of the isocentric C-arm 60 by the positioning elements 70, a free choice of the center of rotation of the isocentric C-arm 60 within the image plane of FIG. 6 is possible. By means of additional positioning elements 70 with a normal alignment relative to the image plane of FIG. 6 (not illustrated), it is furthermore guaranteed that images can be taken relative to a center of rotation outside the image plane by the medical imaging system 10 according to the invention. This causes the three-dimensional reconstruction volume 40 (not illustrated) formed by the overlapping beam paths to be enlarged.

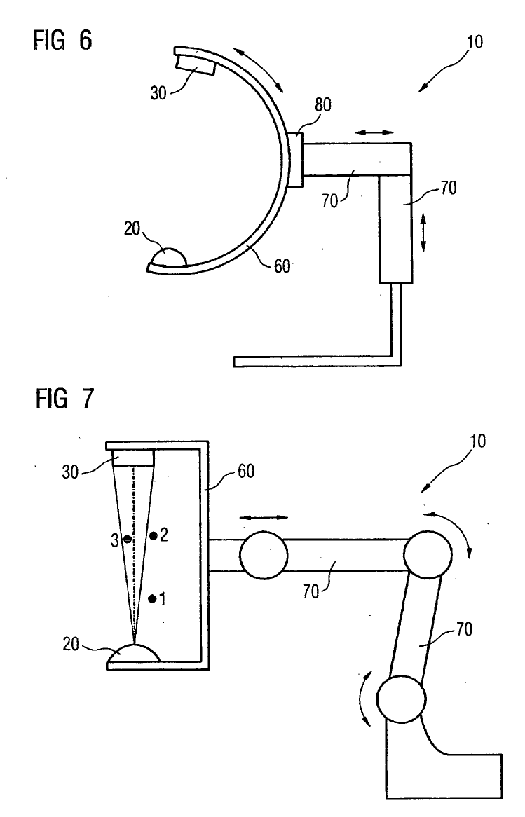

FIG. 7 shows an imaging system 10 according to the invention with an emission source 20 arranged on a U-shaped mounting and a detector 30 correspondingly arranged relative to the center axis of the beam path of the emission source 20. The U-shaped mounting is connected to a first positioning element 70. The first positioning element 70 is connected via a rotably mounted coupling element to a second positioning element 70 so that the positioning elements 70 can take up different angular positions relative to each other. Furthermore, not only the relative position of the positioning elements 70 but also the length of the respective positioning elements 70 can be varied and adjusted as required. The second positioning element 70 is furthermore rotatably connected to a third positioning element 70 by means of a second rotably-mounted coupling element. A control system can, for example by means of stepper motors arranged in the coupling elements, calculate and control the angle of rotation between two positioning elements 70 connected by means of a coupling element.

By means of the relative angular position, in conjunction with the respective length setting as required, of the positioning element 70, a free positioning of the emission source 20 and of the detector 30 is provided on all centers of rotation within the investigation region. For example, three possible centers of rotation 1, 2, 3, the relative positions of which can be chosen as required by a suitable choice of angular positions of the positioning elements 70 are shown in FIG. 7. Combinations of sliding and rotating positioning elements, in conjunction with the use of coupling elements as necessary, guarantee a free positioning of the centers of rotation in a three-dimensional investigation region.

Due to the free positioning capability of the beam paths 90 to 98 (not illustrated) relative to the freely selectable centers of rotation, the medical imaging system 10 guarantees a maximum possible overlap of the beam paths 90 to 98. This makes possible the generation of a larger reconstruction volume 40 (not illustrated) than is possible with the prior art.

1. A method for generating a medical image of an object using a medical imaging system comprising an emission source and a detector arranged relative to each other, comprising:
independently positioning the emission source and the detector relative to each other for a plurality of object irradiations with at least a part of the object being arranged between the emission source and the detector and at least a part of a radiation from the emission source being detected by the detector;
determining a plurality of actual positions of the emission source and the detector for the object irradiations;
obtaining a three-dimensional reconstruction volume of the object in at least one overlap area of at least two of the object irradiations based on the at least two respective actual positions of the emission source and the detector;
calculating a volume image data record of the object from the three-dimensional reconstruction volume;
generating the medical image of the object from the volume image data record relative to a predetermined section plane; and
medically examining the object using the medical image.

2. The method as claimed in claim 1, wherein an image distortion due to an offset position of the emission source relative to the detector is considered during an image data processing.

3. The method as claimed in claim 1, wherein a distortion in superpositioning image data of the object irradiations for obtaining the three-dimensional reconstruction volume is considered during an image data processing.

4. The method as claimed in claim 1, wherein the three-dimensional reconstruction volume and the volume image data record are algebraically depicted as a part of a projection matrix.

5. The method as claimed in claim 4, wherein the projection matrix depicts a complete volume of the object.

6. The method as claimed in claim 1, wherein the three-dimensional reconstruction volume has a same size of the object and is obtained in an overlap area of the object irradiations by positioning the emission source and the detector relative to each other for the object irradiations.

7. The method as claimed in claim 1, wherein at least two three-dimensional reconstruction volumes of the object are obtained in at least two overlap areas of the object irradiations by positioning the emission source and the detector relative to each other for the object irradiations.

8. The method as claimed in claim 7, wherein at least two volume image data records are calculated from the at least two three-dimensional reconstruction volumes.

9. The method as claimed in claim 8, wherein the at least two volume image data records are combined by a graphic image data processing method into one complete date record.

10. The method as claimed in claim 9, wherein the graphic image data processing method is an image stitching method.

11. The method as claimed in claim 7, wherein the at least two three-dimensional reconstruction volumes are combined by a graphic image data processing method into a complete three-dimensional reconstruction volume.

12. The method as claimed in claim 11, wherein a complete volume image data record is calculated from the complete three-dimensional reconstruction volume.

13. The method as claimed in claim 12, wherein the graphic image data processing method is an image stitching method.

14. The method as claimed in claim 1, wherein the emission source is an x-ray radiation source and the detector is an x-ray radiation receiving device.

15. A medical imaging system for generating a medical image of a patient, comprising:
an emission source that generates a beam of radiation;
a detector that detects the radiation penetrating the object to generate the medical image; and
a positioning device that arranges a relative position and alignment of the emission source and the detector to a freely selectable center of rotation.

16. The medical imaging system as claimed in claim 15, wherein the emission source and the detector are arranged on a C-arm and the C-arm can be moved vertically or horizontally by the positioning device.

17. The medical imaging system as claimed in claim 15, wherein the positioning device comprises at least two positioning elements that are connected to each other by at least one rotatably mounted coupling element so that a free positioning of the emission source and the detector relative to the freely selectable center of rotation is guaranteed.

18. The medical imaging system as claimed in claim 15, wherein at least a part of the radiation strikes the detector by arranging the emission source and the detector relative to each other.
